(19) United States
(12) Patent Application Publication
(10) Pub. No.: US 2007/0065449 A1
(43) Pub. Date: Mar. 22, 2007

(54) Method of treating cancer, especially soft tissue sarcoma utilizing gemcitabine in combination with docetaxel and anti-VEGF therapy (bevacizumab)

(76) Inventor: Claire Verschraegen, Albuquerque, NM (US)

Correspondence Address: COLEMAN SUDOL SAPONE, P.C., 714 COLORADO AVENUE, BRIDGE PORT, CT, 06605-1601, US

(21) Appl. No.: 11/522,687

(22) Filed: Sep. 15, 2006

Related U.S. Application Data
(60) Provisional application No. 60/717,652, filed on Sep. 16, 2005.

(51) Int. Cl. A61K 39/395; A61K 48/00; A61K 31/7072; A61K 31/337

(52) U.S. Cl. 424/155.1; 514/49; 514/449; 514/44

(57) ABSTRACT

The present invention relates to a pharmaceutical cocktail, in particular, effective amounts of gemcitabine, in combination with effective amounts of docetaxel and angiogenesis inhibitor, especially a vascular endothelial growth factor (VEGF) inhibitor, such as bevacizumab for the treatment of cancer, in particular sarcoma, especially soft tissue sarcoma. Pharmaceutical compositions and methods of treating cancer, including sarcoma, especially soft tissue sarcoma (prolonging the patient's life, eliminating the tumor, improving the patient's quality of life, shrinking the tumor, prolonging survival and/or preventing the tumor's metastases) are additional aspects of the present invention.

### RELATED APPLICATIONS

This application claims the benefit of priority of provisional application Ser. No. 717,652, filed Sep. 16, 2005, the entire contents of which are incorporated by reference herein.

### FIELD OF THE INVENTION

The present invention relates to a pharmaceutical cocktail, in particular, a combination of effective amounts of gemcitabine, in combination with effective amounts of docetaxel and an angiogenesis inhibitor, including a vascular endothelial growth factor (VEGF) inhibitor such as bevacizumab for the treatment of cancer, in particular sarcoma, especially soft tissue sarcoma (including operable, inoperable or metastatic disease). Pharmaceutical compositions and methods of treating soft tissue sarcoma (eliminating the tumor, shrinking the tumor, prolonging the life of the patient, increasing quality of life by decreasing the grade of adverse events seen with other sarcoma treatments, and/or preventing/reducing the likelihood of the tumor's metastases) are additional aspects of the present invention. In addition, the present invention may be used to favorably impact the therapeutic result (increase the life expectancy at least two years) of patients who have not responded to alternative, traditional anti-cancer therapy.

### BACKGROUND OF THE INVENTION

Sarcomas are rare malignant tumors arising from the mesenchymal tissues at all body sites. Approximately 11,120 new cases are diagnosed each year in the United States; this represents less than one percent of the 1,368,030 new malignant tumors diagnosed each year [1]. Of the 4,960 patients with sarcoma who die annually, many were initially diagnosed years previously. The risk of dying from disease is related to many factors, including the biologic aggressiveness of the disease, its location (ie, the feasibility of management with locoregional techniques, such as surgery or radiation therapy), and comorbid conditions in the patient.

Soft tissue sarcomas (STS) represent a very heterogeneous family of tumors which derive from mesenchymal cells. These tumors appear to arise from malignant precursor cells which can then differentiate along one or several lineages, such as muscle, adipose, fibrous, cartilage, or vascular tissue. Their classification, insights into molecular pathogenesis, and definition of optimal treatment strategies have evolved considerably over the past 10 to 25 years. There are more than 80 histologic subtypes of STS, many of which are associated with distinctive clinical profiles that may have important implications for therapy and prognosis. The small number of cases seen and the great diversity in anatomic site, histopathology, and biology complicate understanding of the natural history of these tumors and their response to diverse therapies.

Although there is a small peak in incidence in early childhood (accounted for primarily by embryonal rhabdomyosarcomas), STS as a whole increase in frequency with advancing age and are most common in patients over age 50. These tumors arise most often in the limbs (particularly the lower extremity), followed in order of frequency by the abdominal cavity/retroperitoneum, trunk/thoracic region and the head and neck.

Local complications from primary or recurrent sarcomas can cause significant morbidity and occasional mortality; however, the most life-threatening aspect of sarcomas is their propensity for hematogenous dissemination, the site of which varies with the type of tumor.

For sarcomas involving the extremity, chest wall, head or neck, the primary metastatic site is the lung; isolated pulmonary metastases occur in 50 to 73 percent [2,3]. As with any clinical rule, this is not a perfect predictive factor, and exceptions are known, such as myxoid liposarcoma and epithelioid sarcomas, which often metastasize first to locoregional soft tissue sites [4].

For retroperitoneal and visceral sarcomas, the primary site of failure is local, either within the peritoneal cavity or the retroperitoneum, and less commonly, the liver [5]. Lymph nodes are rarely involved except in cases of clear cell, epithelioid, and rhabdomyosarcoma [6].

### BRIEF DESCRIPTION OF THE INVENTION

The present invention relates to a method of treating cancer, in particular, sarcoma, especially soft tissue sarcoma, utilizing combination anticancer therapy. In particular, the method is directed to the administration of gemcitabine, docetaxel and a vascular endothelial growth factor (VEGF) inhibitor such as becizumab, in effective amounts to eliminate, inhibit the growth and/or spread of cancer, in particular soft tissue sarcoma in a patient in need thereof. This may be done through coadministration of the three agents at once or through coadministration of gemcitabine and docetaxel, followed by administration of a vascular endothelial growth factor inhibitor such as becizumab within 1 day (24 hours) to about 7 days before or after the coadministration of gemcitabine and docetaxel. The three agents preferably are administered every 2 weeks over a period ranging from about eight (8) to about sixteen (16) weeks in case of operable disease, and indefinitely, or until the cancer stops responding in case of inoperable disease

The present invention also relates to pharmaceutical compositions which comprise a mixture of effective amounts of gemcitabine, docetaxel and an angiogenesis inhibitor, in particular a VEGF inhibitor such as becizumab or other VEGF inhibitor, optionally in combination with a pharmaceutically acceptable carrier, additive or excipient.

Cancers which can be treated using the present invention include, for example, sarcomas, in particular, soft tissue sarcomas. In preferred aspects soft tissue sarcomas which may be treated using the present method include for example, fibrosarcoma, malignant fibrous hystiocytoma, liposarcoma, rhabdomyosarcoma, leiomyosarcoma, hemangiosarcoma, Kaposi's sarcoma, lymphangiosarcoma, synovial sarcoma, neurofibrosarcoma, extraskeletal chrondrosarcoma, extraskeletal osteosarcoma, embryonal sarcoma, alveolar sarcoma, dermatofibrosarcoma, infantile heamangiopericytoma, malignant peripheral nerve sheath tumors (also known as neurofibrosarcomas, malignant schwannomas, and neurogenic sarcomas), alveolar soft part sarcoma, extraskeletal myxoid chondrosarcoma, and extraskeletal mesenchymal sarcoma, among others.

In certain aspects of the present invention, favorable outcomes as otherwise disclosed herein may occur, compared to “traditional anticancer therapy” for soft tissue sarcoma (traditional anticancer therapy is definied herein as a combination of effective amounts of doxorubin and ifosfamide or epirubicin and ifosfamide), wherein the survival of the cancer patient (having at least one form of soft tissue sarcoma, in certain instances metastatic soft tissue sarcoma) may be at least about a year longer, at least about a year a half longer, at least about 2 years longer than a patient receiving traditional anticancer therapy. This is an unexpected result.

### DETAILED DESCRIPTION OF THE INVENTION

The following terms shall be used throughout the specification to describe the present invention.

The term “patient” or “subject” is used throughout the specification to describe an animal, generally a mammal and preferably a human, to whom treatment, including prophylactic treatment, with the compositions according to the present invention is provided. For treatment of those infections, conditions or disease states which are specific for a specific animal such as a human patient, the term patient refers to that specific animal.

The term “neoplasia” or “cancer” is used throughout the specification to refer to the pathological process that results in the formation and growth of a cancerous or malignant neoplasm, i.e., abnormal tissue that grows by cellular proliferation, often more rapidly than normal and continues to grow after the stimuli that initiated the new growth cease. Malignant neoplasms show partial or complete lack of structural organization and functional coordination with the normal tissue and most invade surrounding tissues, metastasize to several sites, and are likely to recur after attempted removal and to cause the death of the patient unless adequately treated. As used herein, the term neoplasia is used to describe all cancerous disease states and embraces or encompasses the pathological process associated with malignant hematogenous, ascitic and solid tumors. Representative cancers include, for example, stomach, colon, rectal, liver, pancreatic, lung, breast, cervix uteri, corpus uteri, ovary, prostate, testis, bladder, renal, brain/CNS, head and neck, throat, Hodgkin's disease, non-Hodgkin's lymphoma, multiple myeloma, leukemia, melanoma, acute lymphocytic leukemia, acute myelogenous leukemia, Ewing's sarcoma, small cell lung cancer, choriocarcinoma, rhabdomyosarcoma, Wilms' tumor, neuroblastoma, hairy cell leukemia, mouth/pharynx, oesophagus, larynx, kidney cancer and lymphoma, among others, including soft tissue sarcomas, which may be treated by the combination of compounds according to the present invention. Sarcomas and in particular, soft tissue sarcoma, as otherwise described herein are particularly preferred targets of the therapeutic method of the present invention.

The term “sarcoma” refers to a type of cancer, in particular, a connective tissue neoplasm, which is usually highly malignant, which is formed by proliferation of mesodermal cells.

The term “soft tissue sarcoma” refers to a type of cancer, especially a type of sarcoma which, in preferred aspects, represents the therapeutic target of the present invention. Soft tissue sarcomas are cancerous (malignant) tumors that originate in the soft tissues of a patient's body. Soft tissues connect, support and surround other body structures. The soft tissues include muscle, fat, blood vessels, nerves, fibrous tissue surrounding joints including tendons and the lining of your joints (synovial tissues) or deep skin tissue. A large variety of soft tissue sarcomas can occur in these areas. They can occur in any part of the body.

Soft tissue sarcomas occur slightly more often in men than in women, and they sometimes occur in children and adolescents. Although there are various types of soft tissue sarcoma, they generally share similar characteristics, produce similar symptoms and are treated in similar ways. Soft tissue sarcomas can arise almost anywhere in the body. About 50 percent occur in the extremities (the arms, legs, hands, or feet), 40 percent occur in the trunk (chest, back, hips, shoulders, and abdomen), and about 10 percent occur in the head and neck.

In their early stages, soft tissue sarcomas usually do not cause symptoms. Because soft tissue is relatively elastic, tumors can grow rather large, pushing aside normal tissue, before they are felt or cause any problems. The first noticeable symptom is usually a painless lump or swelling. As the tumor grows, it may cause other symptoms, such as pain or soreness, as it presses against nearby nerves and muscles. The following Tables A and B below show the types of soft tissue sarcoma which may be treated using the invention of the present application.

Soft tissue sarcomas which may be treated using the present invention include, fibrosarcoma, malignant fibrous hystiocytoma, liposarcoma, rhabdomyosarcoma, leiomyosarcoma, hemangiosarcoma, Kaposi's sarcoma, lymphangiosarcoma, synovial sarcoma, neurofibrosarcoma, extraskeletal chrondrosarcoma, extraskeletal osteosarcoma, embryonal sarcoma, alveolar sarcoma, dermatofibrosarcoma, infantile heamangiopericytoma, malignant peripheral nerve sheath tumors (also known as neurofibrosarcomas, malignant schwannomas, and neurogenic sarcomas), alveolar soft part sarcoma, extraskeletal myxoid chondrosarcoma, and extraskeletal mesenchymal sarcoma.

TABLE A
Major Types of Soft Tissue Sarcomas in Adults

| | | Usual Location in |
|---|---|---|
| Tissue of Origin | Type of Cancer | the Body |
| Fibrous tissue | Fibrosarcoma | Arms, legs, trunk |
| | Malignant fibrous | Legs |
| | hystiocytoma | |
| Fat | Liposarcoma | Arms, legs, trunk |
| Muscle | | |
| Striated muscle | Rhabdomyosarcoma | Arms, legs |
| Smooth muscle | Leiomyosarcoma | Uterus, digestive |
| | | tract |
| Blood vessels | Hemangiosarcoma | Arms, legs, trunk |
| | Kaposi's sarcoma | Legs, trunk |
| Lymph vessels | Lymphangiosarcoma | Arms |
| Synovial tissue | Synovial sarcoma | Legs |
| (linings of joint cavities, | | |
| tendon sheaths) | | |
| Peripheral nerves | Neurofibrosarcoma | Arms, legs, trunk |
| Cartilage and bone- | Extraskeletal | Legs |
| forming tissue | chondrosarcoma | |
| | Extraskeletal | Legs, trunk (not |
| | osteosarcoma | involving the bone) |

TABLE B
Major Types of Soft Tissue Sarcomas in Children

| Tissue of Origin | | Type of Cancer | Usual Location in the Body | Most common ages |
|---|---|---|---|---|
| Muscle | Striated | Rhabdomyosarcoma | | |
| | muscle | Embryonal | Head and neck, genitourinary | Infant-4 years |
| | | Alveolar | Arms, legs, head and neck | Infant-19 years |
| | Smooth | Leiomyosarcoma | Trunk | 15-19 years |
| | Muscle | | | |

| | | | |
|---|---|---|---|
| Fibrous Tissue | Fibrosarcoma | Arms and legs | 15-19 years |
| | Malignant fibrous | Legs | 15-19 years |
| | histiocytoma | | |
| | Dermatofibrosarcoma | Trunk | 15-19 years |
| Fat | Liposarcoma | Arms and Legs | 15-19 years |
| Blood Vessels | Infantile | arms, legs, trunk head | Infant-4 years |
| | hemangiopericytoma | and neck | |
| Synovial tissue | Synovial sarcoma | legs, arms and trunk | 15-19 years |
| (linings of joint cavities, | | | |
| tendon sheaths) | | | |
| Peripheral nerves | malignant peripheral | arms, legs and trunk | 15-19 years |
| | nerve sheath tumors | | |
| Muscular nerves | alveolar soft part | arms and legs | Infant-19 years |
| | sarcoma | | |
| Cartilage and | Extraskeletal myxoid | legs | 10-14 years |
| bone-forming tissue | chondrosarcoma | | |
| | Extraskeletal mesenchymal | legs | 10-14 years |

The term “effective” or “effective amount” means an amount of a compound which is used to effect an intended result. In the present application, the favorable treatment of cancer, in particularly a sarcoma such as a soft tissue sarcoma is the intended effect, manifest in a remission or shrinkage of the cancer/tumor and/or the prevention or a reduction in or the likelihood of the spread (metastases) of the cancer and a substantial increase in the time of survival. The present method will result in an increase in survival of a patient diagnosed with cancer to at least about 1.5 times, at least about 2 times, at least about 2.5 times, at least about 3 times, at least about 3.5 times, at least about 4 times, at least about 5 times, at least about 6 times, at least about 7 times, at least about 8 times, at least about 9 times and at least about 10 times or more the length of time of survival of the untreated patient determined from the time the cancer is diagnosed in the patient. Optimally, the present invention will result in the improvement of the well being of the patient, a shrinkage of the tumor, a prolongation of survival, the remission of cancer and the prevention (as a manifestation of a reduced likelihood or prevention) of metastases of the cancer to other areas of the patient's body. In general, effective amounts of each of the compounds used in the combined therapy according to the present invention include:

Gemcitabine—between about 100 mg and 2.5 grams, preferably about 500 mg to about 2000 mg, preferably about 800 mg, about 1000 mg or about 1500 mg/mm²;

Docetaxel—between about 10 and 100 mg, preferably about 30 to 75 mg, more preferably about 50 mg/m².

Bevacizumab (which may be coadministered with the gemcitabine and docetaxel, or within a week before or after chemotherapy), is administered intravenously, at about 1 mg/kg to about 15 mg/kg, preferably about 5 mg/kg.

The above combination is preferably administered once about every one-two weeks (preferably about every two weeks twice with each course—one course equals 2 dosages—(preferably a total of 6 courses) preferably being administered over a 4-8 week period (preferably over 4 weeks), although the regimen may be administered more frequently depending upon the disease state. Of course, further courses of the combination therapy may be given, as the disease state merits. The dosage of each of the components may be modified to reflect the size and weight of the patient, as well as the severity of the disease state to be treated.

In preferred aspects of the present invention, the combined therapy described above is administered once every two weeks for a total of 12 dosages. The components are preferably co-administered, although it is sometimes desirable to administer the bevacizumab (anti-VEGF therapy) within one week of the gemcitabine and docetaxel administration.

In additional aspects of the present invention, the premedications dexamethasone, at about 5-10 (preferably 8 mg) mg every 12 hours for six doses (three days) and/or zofran (5-10 mg, preferably 8 mg IV) are administered in effective amounts prior to chemotherapy and then intermittently during further therapy pursuant to physician discretion. The dosage schedules according the present invention are referred to herein as low dose, frequent administration.

The term “remission” or “clinical benefit remission” is used to describe a remission in a patient's cancer which may be a complete remission, a partial remission or evidence of stability of the disease.

The term “traditional anticancer therapy” refers to anticancer therapy which is presently used for the treatment of soft tissue sarcoma and preferably is directed to a combination of effective amounts of doxorubicin (60-75 mg/m² per cycle of every three weeks) and ifosfamide (2-10 g/m² for four hours daily for three days) or epirubicin and ifosfamide (same dosage as for doxorubicin and ifosfamide), wherein the survival of the cancer patient (having at least one form of soft tissue sarcoma, in certain instances metastatic soft tissue sarcoma) may be at least about a year longer, at least about a year a half longer, at least about 2 years longer, at least about 2 and a half years longer, at least about 3 years longer, at least about 3 and a half years longer, than a patient receiving traditional anticancer therapy. This is an unexpected result.

The term “coadministration” or “combination therapy” is used to describe a therapy in which at least two active compounds or compositions in effective amounts (in the present application, at least gemcitabine and docetaxel are coadministered with the angiogenesis inhibitor, preferably bevacizumab also being coadministered or being administered before or after the administration of gemcitabine and docetaxel) to treat cancer, in particular sarcomas, especially soft tissue sarcomas, and preferably all three compounds are used to treat a disease state or condition as otherwise described herein at the same time. Although the term coadministration preferably includes the administration of at least two active compounds (gemcitabine and docetaxel) to the patient at the same time, it is not necessary that the compounds be administered to the patient at the same time, although effective amounts of the individual compounds will be present in the patient at the same time.

The term “angiogenesis inhibitor”, “vascular endothelial growth factor inhibitor” “VEGF inhibitor” or “anti-VEGF therapy” all used within context, refers to a compound, composition or therapy which inhibits or otherwise prevents the angiogenesis effects of vascular endothelial growth factor (VEGF, a factor which is involved in the angiogenesis of tissue, including growth in and vascularization of tumors), regardless of mechanism. VEGF inhibitors or anti-VEGF therapy may involve binding of an agent to VEGF to prevent its modulation of a receptor such as VEGFR-1 (flt-1), VEGFR-2 (flk-1 or KDR), or through inhibition of tyrosine kinase in promoting angiogenesis or it may inhibit the binding of VEGF to one or more of its receptors by any one or more mechanisms. Regardless of the mechanism of action, anti-VEGF activity associated with the use of an effective amount of a VEGF inhibitor in the present invention results in a reduction in VEGF activity (angiogenesis/vascularization) in the tumor, and a response which is inhibitory to cancer growth, elaboration and metastases and which helps to promote cancer remission in combination with the other agents. Bevacizumab is a preferred VEGF inhibitor for use in the present invention. Compounds/compositions according to the present invention which represent anti-VEGF therapy (angiogenesis inhibitors) include for example, ZD6474, ZD 6126, AZD2171 (Astra Zeneca), SU6668 and SU5416 (Sugen), bevacizumab (Avastatin), mv833, anti-FLT-1 ribozyme (Angiozyme), and the tyrosine kinase inhibitors SU5416 (Semaxanib), PTK 787 (ZK 222584), ZD4190, ZD6474, CEP-7055, SU11248 and mixtures thereof.

Administration of the compounds according to the present invention is by preferably intravenous injection (preferably, by i.v. drip or i.v. infusion over a period of about 15 minutes to 90 minutes or longer), either through the same syringe/infusion needle or alternatively in up to three separate syringes (for each of the compounds administered to the patient) in effective amounts for treating cancer. As stated, gemcitabine and docetaxel are coadministered to the patient either simultaneously or using two separate syringes/infusion needles seriatim. The administration is generally by iv administration (iv drip or iv infusion) in saline. The VEGF inhibitor (also referred to as an anti-VEGF therapeutic agent), preferably bevacizumab in effective amounts, is administered by iv injection or iv infusion at approximately the same time (within about 90 minutes or so) as the administration of gemcitabine and docetaxel before or after administration of gemcitabine and docetaxel (from about two-three hours (or from about 12 hours) to about 7 days before or after administration of gemcitabine and docetaxel). Gemcitabine and doxetaxel are agents which shrink tumors, bevacizumab both shrinks tumors and prevents or substantially reduces angiogenesis in the tumor body, cutting off blood supply to the tumor. The present method is used to inhibit the growth of cancer, shrink the tumor and/or reduce the likelihood of metastases of the cancer. In a majority of cases, the life expectancy of the patient after therapy is 2 years, 3 years, 4 years, 5 years or more (statistical average).

Therapy of Soft Tissue Sarcoma (STS)

An optimal multidisciplinary approach to the management of advanced STS must recognize the potential benefits and limitations of the available treatment options. The majority of patients who develop metastatic STS are incurable; however, this fact should not lead to therapeutic nihilism. The median survival after development of distant metastases is 11 to 15 months, although 20 to 25 percent of patients are alive at two to three years [2,7]. The prognostic factors for more prolonged survival appear to differ from the factors predicting response to chemotherapy [7], suggesting that survival is more dependent upon disease biology rather than solely on treatment-associated considerations. It is crucial that potentially curative options be identified in appropriate patients so that the opportunity for cure is not overlooked. In selected patients, resection of pulmonary metastases is feasible, and five-year survival rates range from 25 to 40 percent (see below) [8,9].

In patients with metastatic unresectable disease, judicious use of systemic chemotherapy may be associated with important palliative and possible survival benefits in some patients. Although prolongation of survival has not been documented in appropriately designed, prospective clinical trials, no such trials have compared treatment with supportive care alone in patients with advanced STS. The only trials to compare survival in patients receiving chemotherapy with a “no-treatment” group in a controlled manner have been studies in the adjuvant setting, which may not be relevant to patients with advanced unresectable disease [10]. Nevertheless, it is reasonable to state that for patients with advanced metastatic sarcoma (especially for patients with recurrent disease) palliation of symptoms and prevention of rapid disease progression is often the main objective of treatment.

The natural history of metastatic STS was illustrated in an EORTC report that included more than 2,000 patients with advanced STS who were treated with anthracycline-containing chemotherapy [7]. The median overall survival was approximately one year for the entire cohort, regardless of the specific chemotherapy regimen employed. Importantly, the clinicopathologic variables associated with longer survival were different from those predicting objective responsiveness to chemotherapy. The likelihood of response to chemotherapy was greater in younger patients with higher histologic grade primary tumors, non-leiomyosarcoma histologic subtype, and no liver lesions. In contrast, longer median survival was predicted by a good performance status, low grade histologic differentiation, no liver metastases, and a longer duration from initial diagnosis.

These principles can be applied to therapeutic decision-making in the clinical setting. As an example, for some patients with asymptomatic low grade unresectable disease (eg, low grade intraabdominal LMS), it might be reasonable to follow the patient without active chemotherapy or to offer novel low toxicity biologic treatments. Conversely, for patients with high grade chemotherapy-sensitive tumors such as synovial sarcoma or liposarcoma, early use of chemotherapy to control growth and prevent further clinical deterioration may be preferable.

Histologic subtype and response to chemotherapy—It is increasingly recognized that different histologic subtypes of STS exhibit variable patterns of chemosensitivity. As an example, synovial sarcomas and myxoid liposarcomas are among the more chemotherapy-sensitive subtypes [11, 12]. In particular, liposarcomas tend to be sensitive to doxorubicin-based chemotherapy, and synovial sarcoma appears especially sensitive to aggressively-dosed alkylating agents such as ifosfamide. Other subtypes, such as LMS of uterine origin, endometrial stromal sarcomas, myxofibrosarcoma, dedifferentiated liposarcoma, and malignant peripheral nerve sheath tumors (MPNSTs) exhibit considerable individual variability in their patterns of chemosensitivity.

In contrast, other subtypes, such as gastrointestinal stromal tumors (GIST) and most non-uterine LMS have virtually no chance of a major clinical response from conventional anthracycline-based chemotherapy [7,13]. However, for these chemoresistant tumor types, research and molecular targeting may provide crucial clues as to novel therapeutic strategies. For example, recent data demonstrate the impressive efficacy of the tyrosine kinase inhibitor imatinib (STI-571) for patients with advanced GIST, a disease for which there were previously no highly effective treatment options for metastatic disease. At least one case report also suggests activity for Gleevec in patients with metastatic dermatofibroma protuberans.

Taxanes and Soft Tissue Sarcoma

The Southwest Oncology Group (SWOG) reported a trial of paclitaxel in patients with previously untreated advanced soft tissue sarcomas, revealing a response rate of 12.5%.[14] In a recently published phase II trial paclitaxel was evaluated in patients with recurrent or advanced leiomyosarcoma of the uterus. Fifty-three patients were entered and 48 patients were evaluable for toxicity and response. Fifteen patients had received prior irradiation and only 39 patients had been treated with first-line chemotherapy. A median of two (range 1-12) courses of paclitaxel were applied. Four (8.4%) patients had a complete or partial response and 22.9% patients had a stable disease demonstrating that single-agent paclitaxel has modest activity in previously treated uterine leiomyosarcoma.[15] Furthermore, on the basis of phase II trial results, paclitaxel is highly effective in AIDS-related Kaposi's sarcoma as well as in angiosarcoma of the scalp or face. Fata et al.[16] reported eight of nine patients responding to paclitaxel (four partial responses and four clinical complete responses).

Reports from an EORTC phase II study and from an Austrian group on docetaxel have described five (17%) and four (15%) partial responses in 29 and 27 evaluable patients, respectively. Patients had been previously treated with chemotherapy [17,18]. Another ECOG trial investigating docetaxel in previously untreated patients with soft tissue sarcomas reported a low response rate of 5.9% (95% CI 0.1, 29) [19, 23].

Gemcitabine and Soft Tissue Sarcoma

While three trials found no substantial activity of gemcitabine in advanced soft tissue sarcomas,[21-22] an MD Anderson Cancer Center trial demonstrated a remission rate of 18% with a median duration of 3.5 months in patients with soft tissue sarcomas. However, no objective responses were seen in 17 patients with GIST. A recently published trial of the ECOG (E1797) achieved a 4% rate of remissions in previously untreated patients (90% CI 0, 18), but a partial response in a patient lasted for 8 months.[24] By reviewing all trials, activity was particularly observed in patients with angiosarcomas, non-gastrointestinal leiomyosarcoma or unclassified sarcomas. During a phase II trial at the sarcoma centre in Tuebingen, Germany, 19 patients were included to receive gemcitabine 1.0 g/m² as a 30-minute infusion on days 1, 8 and 15 every 4 weeks [25]. All eligible patients had progressive disease during or shortly after an anthracycline/ifosfamide-based regimen. Four of these 19 patients did not start study treatment because of fulminant tumor progression. In the 15 patients who are assessable, a total of 62+ cycles have been applied to date (median 3, range 1-18+). The remission rate was 6% and 47% of patients achieved disease stabilization. The median progression-free survival rate was 3 months (range 1-18+). Eighty-seven percent of the cycles have been applied without any dose modification or delay. This series confirmed the earlier observation of gemcitabine activity in pretreated adult soft tissue sarcoma patients. The calculated progression-free survival rates at 3 and 6 months were 46.7% (95% CI 21.4, 71.9) and 13.3% (95% CI 0, 30.5) [25]. Considering this criteria as the primary endpoint for phase II trials in soft tissue sarcomas,[50] gemcitabine appears to have an efficacy comparable with dacarbazine.[25]

Combination Regimen with Gemcitabine and Taxotere (Docetaxel)

Patients with unresectable leiomyosarcoma of the uterus (n=29) or other primary sites (n=5) were enrolled onto a phase II study of gemcitabine 900 mg/m² intravenously on days 1 and 8 plus docetaxel 100 mg/m² i.v. on day 8 with granulocyte colony-stimulating factor given subcutaneously on days 9 to 15, delivered every 21 days. Patients with prior pelvic radiation received 25% lower doses of both agents. Gemcitabine was delivered over 30 or 90 minutes in cycles 1 and 2 and by 90-minute infusion in all subsequent cycles. Pharmacokinetic studies assessed in vivo differences in gemcitabine concentrations with different rates of infusion. Thirty-four patients (median age, 55 years; range, 32 to 74 years) were enrolled. Fourteen had received prior pelvic radiation. Sixteen of 34 patients had progressed after doxorubicin-based therapy; 18 had no prior chemotherapy. Among 34 patients, complete response was observed in three patients and partial response in 15, for an overall response rate of 53% (95% confidence interval, 35% to 70%). Seven patients had stable disease. Fifty percent of patients previously treated with doxorubicin responded. Hematologic toxicity was common (neutropenia: grade 3, 15%; grade 4, 6%; thrombocytopenia: grade 3, 26%; grade 4, 3%), but neutropenic fever (6%) and bleeding events (0%) were rare. The median time to progression was 5.6 months (range, 4 to 10 months). Gemcitabine plus docetaxel is tolerable and highly active in treated and untreated patients with LMS [26].

TABLE I
Salvage chemotherapy in patients with soft tissue sarcoma refractory
to anthracyclines

| | No. of | | | |
|---|---|---|---|---|
| | evaluable | | | |
| Drug | patients | CR/PR(%) | NC (%) | Reference |
| Docetaxel | 29 | 17 | | 17/ |
| Docetaxel | 27 | 15 | 15 | 18 |
| Gemcitabine | 18 | 11 | 33 | 20 |
| Gemcitabine | 32 | 3 | | 21 |
| Gemcitabine | 29 | 3 | | 22 |
| Gemcitabine | 21 | 4 | 32 | 24 |
| Gemcitabine | 15 | 6 | 47 | 25 |
| Gemcitabine | 18 | 6 | 39 | 27 |
| Docetaxel + Gemcitabine | 34 | 53 | 20.5 | 26 |

Antiangiogenesis and Soft Tissue Sarcoma

Vascular endothelial growth factor (VEGF) is a potent tumor-produced angiogenic factor. The incidence of HER-2/neu, VEGF and CD117 overexpression in soft tissue sarcomas (STS) was determined on the tissue of patients diagnosed with STS during the period of 1986-2001. HER-2/neu overexpression was measured in these patients by immunohistochemistry (IHC) using the Hercep test developed by DAKO. VEGF expression was detected by the avidin-biotin-complex method using Santa Cruz biotechnology (SC 7629). Immunohistochemical staining for c-kit was performed using a 1:250 dilution of the rabbit polyclonal antibody A4502 (IMPATH, CA) with the EnVision detection system. Overexpression was correlated with survival in patients with specific histological subtypes of STS. Two hundred and seventy three patients were diagnosed as having STS between 1986 and 2001, however of these patients, only 90 (51 females and 49 males) had enough sample available for testing. Patients who overexpressed VEGF had a significantly shorter survival (23 vs. 52 months; p=0.01). There was no effect of overexpression of either CD117 or HER-2/neu on survival. Per individual histological, overexpression in malignant fibrous histiocytoma of either VEGF or CD117 increased survival (41.3 vs. 19.5 months, p=0.01; and 84.5 vs. 17 months, p=0.006 respectively). In leiomyosarcoma, VEGF overexpression significantly decreased survival (7.5 vs. 76 months, p=0.03), while CD117 overexpression significantly increased survival (70.9 vs. 46.3 months, p=0.03). VEGF overexpression is associated with an adverse outcome in STS. Whether this is true of any particular histological subtype is unclear and needs further investigation. Site-specific agents targeting the VEGF bio-markers (alone or with conventional therapy) may have a therapeutic role and need to be evaluated in clinical trials [28].

In another study, the serum levels of VEGF were measured before treatment and during follow-up in patients undergoing primary treatment for suspected soft tissue sarcoma (STS) to assess the value of serum VEGF as a tumor marker. Between April 2001 and September 2002, serum VEGF levels were analyzed prospectively in 144 patients undergoing primary treatment (surgery, 123; cytotoxic chemotherapy, 10; oral imatinib, ei8ght; radiotherapy, 3) for soft tissue sarcoma. Serum VEGF was measured by immunoassay before treatment, in the immediate postoperative interval in patients undergoing surgery, and during follow-up. Serum VEGF concentrations were also measured in 15 healthy control volunteers. Median pretreatment serum VEGF levels were significantly raised in patients with grade 2 and grade 3 sarcomas compared with concentrations in patients with benign lesions (413 and 467 versus 233 pg/ml respectively; P=0.007 and P=0.003 respectively). In patients with tumors that had a high level of VEGF expression before treatment, follow-up measurements reflected disease status after treatment. Serum VEGF expression correlated with grade in soft tissue sarcoma and reflected response to treatment [29]. The concept of VEGF inhibition is currently investigated in untreated or previously treated patients using bevacizumab combined with doxorubicin in one US on-going phase II trial. This combination was found to be toxic for the heart, with bevacizumab enhancing the deleterious effect of doxorubicin on the myocardium.

Docetaxel (Taxoter®) (RP56976) (NSC-628503)

a. Description

Origin

In the late 1960's, the National Cancer Institute large-scale plant screening program found that a crude extract of the bark from the Pacific yew, Taxus brevifolia, had activity against the P388 mouse leukemia. In 1971, Wani, Taylor et al. isolated and characterized paclitaxel (Taxol®), the active principle of the extract. It has become evident that paclitaxel (Taxol®) has activity against several human malignancies including refractory ovarian cancer and breast cancer.

Several years ago, researchers at Rhone-Poulenc Rorer with the cooperation of the French “Centre National de Recherche Scientifique (CNRS)” were able to prepare docetaxel (Taxotere®), a semisynthetic analog of paclitaxel, using a precursor extracted from the needles of the European yew, Taxus baccata, a renewable source. Docetaxel presents as a white powder, either anhydrous or as the trihydrate. In vitro, docetaxel promotes tubulin assembly in microtubules and inhibits depolymerization thus stabilizing microtubules, which is different from the action of other spindle poisons in clinical use. This can lead to bundles of microtubules in the cell, which by blocking cells in the M phase of the cell cycle, results in the inability of the cells to divide. Comparing docetaxel and paclitaxel using the “tubulin in vitro assay”, the concentration required to provide 50% inhibition of microtubule disassembly (or IC50) is 0.2 μM for docetaxel and 0.4 μM for paclitaxel.

b. Toxicology

The major toxic effect of docetaxel, which limits dose, is neutropenia. Other toxic effects, which may be seen, include leukopenia, thrombocytopenia, anemia, asthenia, dysgeusia, myalgia, arthralgia, nail changes and conjunctivitis. Severe anaphylactoid reactions, characterized by a flush associated with hypo- or hypertension, with or without dyspnea, may occur. Other toxicities include cutaneous reactions (e.g., skin rash, desquamation following localized pruriginous maculopapular eruption, skin erythema with edema), hypersensitivity reactions (flushing, pruritis, fever, chills, rigors, lower back pain), dyspnea with restrictive pulmonary syndrome, pleural effusions, arrhythmias, pericardial effusions, fluid retention syndrome, ascites, myopathy, digestive tract toxicities (nausea, vomiting, oral mucositis, diarrhea, anorexia), alopecia, extravasation reaction (erythema, swelling, tenderness, pustules), reversible peripheral phlebitis, peripheral edema, reversible increase in liver function tests, hepatic failure and neurotoxicity (reversible dysesthesias or paresthesias, peripheral neuropathy, seizure, headache, lethargy or somnolence). Patients with SGOT>1.5 times normal and alkaline phosphatase>2.5 times normal appear to have decreased docetaxel clearance and appear to be more likely to suffer severe toxicity, including drug-related death.

c. Pharmacology

Docetaxel is a cytotoxic anticancer drug and, as with other potentially toxic compounds, caution should be exercised when handling and preparing docetaxel solutions. The use of gloves is recommended.

If docetaxel concentrate, initial diluted solution, or final dilution for infusion should come into contact with the skin, immediately and thoroughly wash with soap and water. If docetaxel concentrate, initial diluted solution, or final dilution for infusion should come into contact with mucosa, immediately and thoroughly wash with water.

Docetaxel for Injection Concentrate requires two dilutions prior to administration. Please follow the preparation instructions provided. Note: Both the docetaxel for Injection Concentrate and the diluent vials contain an overfill.

How supplied: Docetaxel for Injection Concentrate is supplied in a single-dose vial as a sterile, pyrogen-free, non-aqueous, viscous solution with an accompanying sterile, non-pyrogenic, diluent (13% ethanol in Water for Injection) vial. The following strengths are available: 80 mg and 20mg strengths are available.

Storage: Store between 2 and 25° C. (36 and 77° F.). Retain in the original package to protect from bright light. Freezing does not adversely affect the product.

Preparation of the initial diluted solution: Gather the appropriate number of vials of docetaxel for Injection Concentrate and diluent (13% Ethanol in Water for Injection). If the vials were refrigerated, allow them to stand at room temperature for approximately 5 minutes. Aseptically withdraw the contents of the appropriate diluent vial into a syringe and transfer it to the appropriate vial of docetaxel for Injection Concentrate. If the procedure is followed as described, an initial diluted solution of 10 mg docetaxel/ml will result. Gently rotate the initial diluted solution for approximately 15 seconds to assure full mixture of the concentrate and diluent. The initial diluted docetaxel solution (10 mg docetaxel/ml) should be clear; however, there may be some foam on top of the solution due to the polysorbate 80. Allow the solution to stand for a few minutes to allow any foam to dissipate. It is not required that all foam dissipate prior to continuing the preparation process. The initial diluted solution may be used immediately or stored either in the refrigerator or at room temperature for a maximum of 8 hours.

Preparation of the final dilution for infusion: Aseptically withdraw the required amount of initial diluted docetaxel solution (10 mg docetaxel/ml) with a calibrated syringe and inject into a 250 ml infusion bag or bottle of either 0.9% Sodium Chloride solution or 5% Dextrose solution to produce a final concentration of 0.3 to 0.74 mg/ml. If a dose greater than 200 mg of docetaxel is required, use a larger volume of the infusion vehicle so that a concentration of 0.74 mg/ml docetaxel is not exceeded. Thoroughly mix the infusion by manual rotation. As with all parenteral products, docetaxel should be inspected visually for particulate matter or discoloration prior to administration whenever the solution and container permit. If the docetaxel for Injection initial diluted solution or final dilution for infusion is not clear or appears to have precipitation, these should be discarded.

Contact of the docetaxel concentrate with plasticized PVC equipment or devices used to prepare solutions for infusion is not recommended. In order to minimize patient exposure to the plasticizer DEHP (di-2-ethylhexyl phthalate), which may be leached from PVC infusion bags or sets, the final docetaxel dilution for infusion should be stored in bottles (glass, polypropylene) or plastic bags (polypropylene, polyolefin) and administered through polyethylene-lined administration sets.

Administration: The drug will be administered to the patients as a 1-hour IV infusion under ambient room temperature and lighting conditions.

Stability: The initial diluted solution may be used immediately or stored either in the refrigerator or at room temperature for a maximum of 8 hours.

Docetaxel infusion solution, if stored between 2 and 25° C. (36 and 77° F.) is stable for 4 hours. Fully prepared docetaxel infusion solution (in either 0.9% Sodium Chloride solution or 5% Dextrose solution) should be used within 4 hours (including the 1 hour IV administration).

Handling and disposal: Procedures for proper handling and disposal of anticancer drugs should be considered. Several guidelines on this subject have been published. There is no general agreement that all of the procedures recommended in the guidelines are necessary or appropriate.

Supplier: This drug is commercially available for purchase by a third party.

Gemcitabine (Gemzar®)

a. Description

Origin

Gemcitabine HCl is a nucleoside analogue that exhibits antitumor activity. It has a molecular weight of 299.66. Gemcitabine HCl is a white to off-white solid. It is soluble in water, slightly soluble in methanol, and practically insoluble in ethanol and polar organic solvents. The clinical formulation is supplied in a sterile form for intravenous use only. Vials of gemcitabine contain either 200 mg or 1 g of gemcitabine HCl (expressed as free base) formulated with mannitol (200 mg or 1 g, respectively) and sodium acetate (12.5 mg or 62.5 mg, respectively) as a sterile lyophilized powder. Hydrochloric acid and/or sodium hydroxide may have been added for pH adjustment.

Chemical name: 2′-deoxy-2′,2′-difluorocytidine monohydrochloride ((beta)-isomer). The empirical formula for gemcitabine HCl is C₉H₁₁F₂N₃O₄.HCl.

b. Toxicology

Myelosuppression is the principal dose-limiting toxicity with Gemcitabine therapy. Nausea and vomiting are commonly reported (69%) but are usually of mild to moderate severity. Gemcitabine has associated with transient elevations of one or both serum transaminases. Mild proteinuria and hematuria were commonly reported. Clinical findings consistent with the hemolytic uremic syndrome (HUS) were reported in 6 of 2429 patients (0.25%) receiving gemcitabine in clinical trials. The overall incidence of fever was 41%, and “flu syndrome” was reported for 19% of patients. Rash was reported in 30% of patients. Edema (13%), peripheral edema (20%) and generalized edema (<1%) were reported. Respiratory failure is rare, but gemcitabine should be discontinued if it happens. For a more complete description of expected adverse events please refer to the agent's package insert.

c. Pharmacology

Preparation Refer to the package insert for standard preparation instructions. Store at controlled room temperature (20° to 25° C.) (68° to 77° F.). To mix, add 5 ml or 25 ml of normal saline to 200 mg or 1000 mg vial and shake to dissolve. This 38 mg/ ml solution can be further diluted in normal saline to give the total drug amount in 100 ml to 250 ml.

Administration: Gemcitabine will be administered intravenously as a short intravenous infusion over 30 minutes. Good clinical practice should be employed when administering the drug. Caution should be utilized when handling and preparing the solution. Therefore, double gloving is required. Dermal contact should be avoided. If gemcitabine solution contacts the skin, immediately wash the area with soap and water, rinsing thoroughly. Needles and syringes should be discarded in puncture resistant containers without being clipped. Gloves and any other contaminated materials should be disposed of in biohazard waste containers, which should be sealed and then incinerated at extremely high temperatures. Small spills should be treated with detergent and water. Wash water and material used to clean the spill should be disposed of in biohazard containers and again sent for incineration.

Bevacizumab (Avastin®) (rhuMAb-VEGF)( Anti-VEGF monoclonal antibody)

a. Description

Origin

Bevacizumab is a recombinant human/murine chimeric monoclonal antibody directed against vascular endothelial growth factor (VEGF).). It is prepared by engineering VEGF-binding residues of a murine anti-VEGF monoclonal antibody into framework regions of human immunoglobulin-1 (IgG1) (Prod Info Avastin, 2004). Only 7% of the amino acid sequence is derived from the murine antibody, with 93% from IgG1 [30].

Human VEGF mediates neoangiogenesis in normal and malignant vasculature; it is overexpressed in most malignancies and high levels have correlated with a greater risk of metastases and poor prognosis in many. When VEGF interacts with its receptor in in vitro models of angiogenesis, endothelial cell proliferation and new blood vessel formation occur. In animal models, VEGF has been demonstrated to induce vascular endothelial-cell proliferation/migration, sustain survival of newly-formed blood vessels, and enhance vascular permeability. Bevacizumab binds and neutralizes all human VEGF forms via recognition of binding sites for the two human VEGF receptor types (flt-1 and flk-1). In animal models, the antibody has been shown to stabilize established tumors or suppress tumor growth by inhibiting angiogenesis induced by VEGF [31].

b. Toxicology

Minor bleeding or hemorrhage (eg, epistaxis, and hemoptysis), and thromboembolic events (eg, deep vein thrombosis) have accompanied administration of bevacizumab in some cancer patients. Other serious but uncommon events included; gastrointestinal hemorrhage, subarachnoid hemorrhage, fatal pulmonary hemorrhage, and hemorrhagic stroke (Prod Info Avastin(™), 2004). Grade ¾ hypertension (12%), deep venous thrombosis (9%), and intra-abdominal thrombosis (3%) occurred in patients receiving bolus irinotecan/5-fluorouracil/leucovorin plus bevacizumab in a trial of patients with untreated metastatic colorectal cancer. Myocardial infarction and hypotension have also been reported. Modest increases in diastolic and systolic blood pressures and clinical hypertension have been reported frequently during bevacizumab therapy (23% to 34% of patients) and may need to be controlled with antihypertensive medications. Mild asthenia and headache have been common during therapy (up to 70% and 50% of patients, respectively), but may be dose-dependent. Dizziness (22%), hypokalemia (14%) and bilirubinemia (4%) vomiting (50%), anorexia (40%), constipation (30%), stomatitis (30%), dyspepsia (20%), weight loss (15%), taste disorder (16%) and flatulence (16%), myalgia (10%), skin ulcer (6%) and confusion (3%) may occur. Grade ¾ diarrhea (30%) and abdominal pain (6%) were also reported. Nausea and vomiting may be more severe with higher doses. Gastrointestinal perforation occurred in 2% of patients receiving bolus irinotecan/5-fluorouracil/leucovorin plus bevacizumab versus 4% of patients receiving 5-fluorouracil/leucovorin plus bevacizumab in a trial of patients with untreated metastatic colorectal cancer; a typical presentation included abdominal pain, constipation, and vomiting [32].

Proteinuria of varying severity or nephrotic syndrome has been described during therapy with bevacizumab [33]. Life threatening or fatal pulmonary hemorrhage occurred in 3 to 1% of patients with squamous cell non-small cell lung cancer (4% nonsquamous cell histology) receiving bevacizumab in combination with chemotherapy compared to 0% in the chemotherapy alone group; these events presented suddenly as major hemoptysis and occurred in patients with cavitation and/or necrosis of the tumor, either preexisting or developing during therapy [34]. Skin rash (type unspecified) has been described in some patients following infusion. Low-grade fever and infection have occurred with variable frequency during therapy. The incidence of immunogenicity with bevacizumab exists, but has not been determined (prod info avastin(™), 2004). No antibodies to bevacizumab were reported in a phase I study (n=25) where patients received four doses of 0.1 to 10 mg/kg over 42 days, and assays were performed for up to 70 days [31]. There is insufficient clinical experience with bevacizumab to confirm its safety in pregnancy.

Black Box Warnings

Gastrointestinal Perforations/Wound Healing Complications

Avastin administration can result in the development of gastrointestinal perforation and wound dehiscence, in some instances resulting in fatality. Gastrointestinal perforation, sometimes associated with intra-abdominal abscess, occurred throughout treatment with Avastin (ie, was not correlated to duration of exposure). The incidence of gastrointestinal perforation in patients receiving bolus-IFL with Avastin was 2%. The typical presentation was reported as abdominal pain associated with symptoms such as constipation and vomiting. Gastrointestinal perforation should be included in the differential diagnosis of patients presenting with abdominal pain on Avastin. Avastintherapy should be permanently discontinued in patients with gastrointestinal perforation or wound dehiscence requiring medical intervention. The appropriate interval between termination of Avastin and subsequent elective surgery required to avoid the risks of impaired wound healing/wound dehiscence has not been determined.

Hemorrhage

Serious, and in some cases fatal, hemoptysis has occurred in patients with non-small cell lung cancer treated with chemotherapy and Avastin. In a small study, the incidence of serious or fatal hemoptysis was 31% in patients with squamous histology and 4% in patients with adenocarcinoma receiving Avastin as compared to no cases in patients treated with chemotherapy alone. Patients with recent hemoptysis should not receive Avastin.

c. Pharmacology

The pharmacokinetics of bevacizumab are linear after doses of 0.3 mg/kg or greater (Anon, 2002). Following 90-minute intravenous infusions of 0.3, 1, 3, and 10 mg/kg in advanced cancer patients (n=25), peak serum concentrations of bevacizumab ranged from 5 to 9 mcg/mL, 21 to 39 mcg/mL, 52 to 92 mcg/mL, and 186 to 294 mcg/mL, respectively; slight accumulation was observed with repeat doses (weekly), but this was not significant and pharmacokinetics remained linear. Steady-state levels of bevacizumab were obtained in 100 days in 491 patients who received 1 to 20 mg/kg weekly, every 2 weeks, or every 3 week Following 90-minute intravenous infusions of 0.3, 1, 3, and 10 mg/kg in advanced cancer patients (n=25), AUC_{inf} values ranged from 31 to 87, 240 to 382, 550 to 1720, and 2480 to 6010 mcg/mL×day, respectively [31]. Central volume of distribution of bevacizumab was greater in males than in females (3.25 L vs. 2.66 L) in 491 patients who received 1 to 20 mg/kg weekly, every 2 weeks, or every 3 week. The clearance of bevacizumab was higher (0.262 L/day vs. 0.207 L/day) in males than females; patients with a higher tumor burden (at or above median value of tumor surface area) also had a higher clearance (0.249 L/day vs. 0.199 L/day). The estimated elimination half-life of bevacizumab was 20 days (range 11 to 50 days) in a pharmacokinetic population analysis of 491 patients receiving 1 to 20 mg/kg weekly, every 2 weeks, or every 3 weeks.

VEGF Serum Level Changes

In advanced cancer patients, free VEGF serum levels were reduced significantly following the first dose of bevacizumab 1 to 10 mg/kg, and remained below the limit of detection for the duration of the study (repeat doses at 28, 35, and 42 days). Levels of total VEGF increased with all doses (0.1 to 10 mg/kg), presumably as a result of increased VEGF synthesis/distribution or reduced VEGF clearance secondary to complex formation (between VEGF and bevacizumab) [31].

Storage And Stability

Store bevacizumab vials protected from light, under refrigeration at 2 to 8 degrees Celsius/36 to 46 degrees Fahrenheit. Do not freeze or shake. This product contains no preservative (Prod Info Avastin(™), 2004).

Diluted solutions of bevacizumab in 100 mL 0.9% Sodium chloride Injection may be stored for up to 8 hours under refrigeration (2 to 8 degrees Celcius/36 to 46 degrees Fahrenheit) (Prod Info Avastin(™), 2004). Early phase I trials were conducted with bevacizumab diluted in 5% Dextrose for Injection. However, results indicate that dextrose inactivates bevacizumab.

Dosage and Administration

The recommended dose of bevacizumab is 5 milligrams/kilogram infused intravenously over 30 minutes every 2 weeks until disease progression diminishes. Bevacizumab should follow chemotherapy. Efficacy of single-agent bevacizumab has not been established. The calculated dose of bevacizumab in 100 milliliters of 0.9% Sodium Chloride Injection should initially be infused over 90 minutes; subsequent doses can be administered in shorter periods of time (60 minutes for the second infusion and 30 minutes for the third infusion, if well-tolerated). Do not administer as an intravenous bolus or push (Prod Info Avastin(™), 2004).

Exemplary Study

Conclusions and Rationale to the Study

Because the combination of gemcitabine and docetaxel has shown activity in soft tissue sarcoma, we hypothesize that the addition of an antiangiogenesis agent may enhance the anticancer activity, as shown in other tumor types.

Patient Eligibility

Inclusion Criteria

All patients, 18 years of age or older, with chemotherapy naive soft tissue sarcoma are eligible if there is measurable disease Prior surgery or radiotherapy for the primary tumor is allowed but needs to have been completed at least 2 weeks from entry, and patient should have completely recovered from the procedures.

Patients must have a life expectancy of at least 12 weeks.

Patients must have a Zubrod performance status of 0-2.

Patients must sign an informed consent.

Patients should have adequate bone marrow function defined by an absolute peripheral granulocyte count of ≧1,500 or cells/mm³ and platelet count >100,000/mm³ and absence of a regular red blood cell transfusion requirement.

Patients should have adequate hepatic function with a total bilirubin <2 mg/dl and SGOT or SGPT<two times the upper limit of normal, and adequate renal function as defined by a serum creatinine ≦1.5×upper limit of normal.

Exclusion Criteria

Patients with symptomatic brain metastases are excluded from this study.

Pregnant women or nursing mothers are not eligible for this trial. Patients of child bearing potential must use adequate contraception.

Patients may receive no other concurrent chemotherapy or radiation therapy during this trial.

Patients with severe medical problems such as uncontrolled diabetes mellitus or cardiovascular disease or active infections are not eligible for this trial.

Treatment Plan

This is a single-arm, non-randomized Phase I/II trial of gemcitabine, docetaxel and bevacizumab. Patients will be entered in a two-step Simon design at level 2, unless there is too much toxicity, as described in the statistical section. In any case, the determination of the Phase II dose will be done on the toxicity observed during the first course of treatment only. Before treating a new cohort, side effects will be critically reviewed to evaluate the toxicity of this regimen.

Dose and Schedule

| | | | Bevacizumab |
|---|---|---|---|
| | | | (administered |
| Drug | Docetaxel | Gemcitabine | after chemotherapy) |
| Starting dose | 50 | 1000 | 5 mg/kg |
| Level 1 | 50 | 1250 | 5 mg/kg |
| Level 2 | 50 | 1500 | 5 mg/kg |
| Level-1 | 40 | 800 | 5 mg/kg |

Combination is administered every 2 weeks. One course is 4 weeks.

Drug Administration:

Drug will be administered as per guidelines defined in the Drug Background Information section

Pre-medications prior to starting treatment will consist of dexamethasone 8 mg every 12 hours for six doses, starting 24 hours prior to chemotherapy administration, and zofran 8 mg IV or PO prior to chemotherapy, then as indicated at physician discretion.

Criteria for Initiating Subsequent Courses.

Unless there is early progression of disease, at least two courses of this combination will be administered to each patient. Repeated doses may be given to the patients who benefit from the treatment (either complete or partial remission, or stabilization of disease) pending complete recovery of non hematologic toxicity to grade ≦1 (aside from alopecia), with return of blood cell counts to ≧1500 granulocytes and ≧100,000 platelets. If myelosuppression or non-myelosuppressive toxic effects precluding therapy persist for more than 2 weeks, patients will be taken off study. Patients that tolerate repeated treatments with no grade >1 toxicity may be treated at a higher dose (see dose modifications).

Dose Modifications:

Blood counts will be drawn every 2 weeks with a leeway of ±2 days and patients will be followed for nonmyelosuppressive toxicity at every doctor visit. A diary will be given to each patient. The next course of the combination may be restarted upon recovery of all toxicity (see 5.4).

Doses will be decreased according to the following chart:

| | | |
|---|---|---|
| Adverse Event at time of | Hold | −1 dose level after recovery |
| redosing | | to ANC ≧1,500 and |
| | | platelets ≧100,000* |
| Granulocytes <1000/mm³ | | X |
| Granulocytes <500/mm³ | Skip dose, then | |
| | reduce by −1 dose | |
| | level | |
| Platelets <100,000/mm³ | | X |
| Platelets <50,000/mm³ | Skip dose, | |
| | then reduce by −1 | |
| | dose level | |
| Non hematologic side | | X |
| effects of grade 3 | | |
| Non hematologic side | X | |
| effects of grade 4 | Check with PI | |

*Check labs and side effect recovery weekly

The lowest level is level −1. If patients cannot tolerate this level, they will be taken off study.

G-CSF may be used while patients are receiving this combination. If myelosuppression occurs and drug is held, G-CSF may be given according to the general guidelines for its use. Do not administer G-CSF concomitantly with this combination.

If pneumonitis grade 2 or higher develops in a given cycle and is related to gemcitabine, gemcitabine should be promptly discontinued and the patient should be removed from protocol treatment. Treatment with corticosteroids should be given according to established guidelines.

The diagnosis of HUS should be considered if the patient develops anemia with evidence of microangiopathic hemolysis as indicated by elevation of bilirubin or LDH, reticulocytosis, severe thrombocytopenia, and/or evidence of renal failure (elevation of serum creatinine of BUN). Gemzar therapy should be discontinued immediately. Renal Failure may not be reversible even with discontinuation of therapy and dialysis may be required.

Pretreatment Evaluation

A complete history and physical examination to include performance status, weight, blood pressure, and concurrent non-malignant disease and therapy will be done before starting treatment. Prior surgery, chemotherapy, and radiotherapy details will be noted.

Laboratory studies should include a CBC with differential cell count, platelet count, urinalysis, chest x-ray, SMA-12, electrolytes, PT/PTT, electrocardiogram, and appropriate tumor marker levels. A baseline imaging study of the target lesions will be performed. Other X-rays will be done as clinically indicated.

Baseline serum VEGF levels and immunocytochemistry for VEGF receptor will be done on the pathology specimen if feasible.

Evaluation During Study

Physical examination, performance status, blood pressure, and toxicity recording will be done before each course of therapy. Vital signs will be taken prior and after each chemotherapy administration. The patient will fill up a diary, and return it at each clinic visit.

During the study, patients will be followed with every 2 week (±2 days) CBC, differential and platelet counts. Chemistries and urinalysis will also be performed before each course within a ±3-day leeway.

Measurable and evaluable disease will be assessed by imaging studies every 2 courses to evaluate tumor response. The same imaging technique must be repeated to follow patients from baseline.

Criteria for Response and Toxicity

Toxicity is evaluated at each course of therapy.

All toxicities encountered during the study will be evaluated according to the grading system (0-4) NCI CTCAE version 3 and recorded prior to each course of therapy. Duration of side effects and ancillary treatment will be recorded. Grade 4 (except hematologic) toxicities should be reported immediately to the Study Chairman, who, in turn, must notify the IRB and the FDA.

Any adverse event will be evaluated and treated by the investigator as deemed appropriate in light of the medical situation. All pertinent observations and treatments will be recorded, both on an outpatient and inpatient basis (should hospital admission be required). Should death occur, on study, every effort will be made to obtain autopsy data with careful examination of suspected target organs such as the bone marrow and lungs, as well as examination of the tumor for possible chemotherapy induced changes.

Tumor Measurements: The RECIST criteria will be used to determine antitumor response. All measurements on imaging studies will be reviewed by the principal investigator.

The RECIST criteria can be found on the following website:
http://www3.nci.nih.gov/bip/RECIST.htm.

Response duration is measured from the time of response (not the beginning of treatment unless there is stable disease) until there is evidence of progressive disease.

Time to progression will be measured from the time of response (not the beginning of treatment unless there is stable disease) until there is evidence of progressive disease.

Criteria for Discontinuing Therapy

Increasing disease during therapy

The development of unacceptable toxicity.

Change in the medical status of the patient such that the investigator believes that patient safety will be compromised.

Non-compliance by the patient with protocol requirements.

Patient refusal.

Statistical Considerations

This is an open-label, Phase I/II study of docetaxel, gemcitabine, and bevacizumab administered to patients with soft tissue sarcoma. The purpose of this Phase I/II study is:
To determine the optimal dose for this combination.
To determine the antitumor activity of this combination administered to patients with soft tissue sarcoma. Furthermore, patients who achieve a response leading to tumor resectability with a possibility for cure will be offer surgery.
To evaluate the quantitative and qualitative toxicities of this combination with this schedule, in this population.

The primary endpoint is overall response rate (complete and partial responses). Activity will be calculated as the proportion of patients with responsive disease and the 95% confidence interval for response calculated. Treatment toxicity and Kaplan-Meier estimates of time to progression and survival will also be determined. Correlation of toxicity, response and demographic factors will be investigated in an exploratory fashion.

Sample Size and Stopping Rule:

In the first part of the study, 3 patients will be accrued at each dose level. The DLT will be based upon the NCI CTCAE version 3 and defined as the dose of drug that produces a reversible grade 4 hematotogical toxicity lasting >7 days, or reversible grade ≧3 (grade 2 for neurotoxicity) nonmyelosuppressive toxicity in >33% of patients treated at a given dose level. The DLT will be based only on patients receiving their first course of treatment. If no DLT is observed among the initial 3 patients placed on a dosage level, the dosage will be escalated for the successive cohort of 3 patients. Only toxicity labeled possible, probable or definite will be taken into account. If one instance of DLT is observed among the initial 3 patients, an additional 3 patients must be treated at that level. If no further instances of DLT are observed, the dosage will be escalated for the next cohort of 3 patients. If, at any time, 2 patients in one cohort develop a DLT, entry onto this cohort will be terminated. The recommended phase II dose will be defined to be the highest dose for which no more than one patient develop a DLT. If 1 patient or less develops a DLT at the +2 Dose Level, this dose will be used to test the activity of the combination. Then, Simon's two-stage optimum design will be employed. The null hypothesis is that the overall response rate is ≦15% vs. the alternative hypothesis that the overall response rate will be ≧40%. At the first stage, 7 evaluable patients will be enrolled. If one or no responses occur, the trial will be terminated; otherwise another 18 evaluable patients will be treated for a total of 25. If the total number of responses are more than or equal to 7 among the 25 evaluable patients, this agent would be considered worthy of further testing. This optimum design has 0.806 probability of concluding that the drug is effective when the real response rate is ≧40% for the targeted power=80%. The actual probability of concluding that this treatment is not effective when it is actually effective is 4.9% for the targeted Type I error=5%. The over all expected sample size is 12.10 with this design. The probability of early termination of trial is 0.717.

It is anticipated that 1 patient will be accrued per month and that a total of 7 to 25 patients will be needed to establish the activity of administering this combination to patients with soft tissue sarcoma.

Data and Protocol Management

Protocol Compliance: The attending physician and oncology research nurse must see each patient prior to each course of treatment. All required interim and pre treatment data should be available prior to each treatment course for the physician to make a designation as to tumor response and toxicity grade.

Data Entry: Available data must be entered into the clinical data management record before a course of therapy can be given. Every effort will be made to obtain missing data to insure all necessary data have been obtained and recorded at completion of the study.

Accuracy of Data Collection: The study chairman will be the final arbiter of response or toxicity should a difference of opinion exist.

At the conclusion of the study, appropriate follow-up care will be provided for all patients.

Analysis of Data

Demographic data will be displayed, and summary statistics will be used to describe the study population (i.e. means and range of age, weight, height, race, numbers of males and females, description of baseline performance status characteristics, tabulation of tumor histologies, when applicable).

The results of the safety and toxicity evaluation will be tabulated using the grading scale of the NCI CTCAE version 3. Efficacy data will also be tabulated.

Pharmacology data will be described by patients.

Reporting Requirements

Any serious and/or unexpected and severe (grade 4 non-hematologic toxicity) toxicity will be reported immediately to the Study Chairman who, in turn, must notify the Chairman of the IRB (CFR 312.32).

Serious adverse events include toxicities which cause death, are life-threatening, result in inpatient hospitalization or prolongation of an existing hospitalization, cause a persistent or significant disability/incapacity, or a congenital anomaly/birth defect. Such complications should be reported in writing to the FDA within 5 working days with the exceptions of unexpected death or life-threatening event, which should be reported within 5 working days, after learning of the event.

Laboratory tests for hematology, chemistry and urinalysis will be performed to assess toxic effects of the study treatment. All laboratory tests performed will be assessed by the investigator as to their clinical significance. Any post-baseline laboratory value which is found to be clinically significant will be assessed by the investigator for a causal relationship to the study drug and the appropriate action taken.

Removal of a patient from the study because of adverse experiences or changes in laboratory test values, whether by the investigator or by the patient's own volition, should be reported promptly to the Study Chairman.

Study site personnel must notify Lilly or its designee immediately of any “serious” (defined below) adverse event experienced by a patient. In addition, adverse events must be reported to regulatory authorities according to the definitions and timelines specified in the local laws and regulations.

Serious events are defined as those that result in:
Death.
Initial or prolonged inpatient hospitalization.
A life-threatening situation (where the patient is at immediate risk of death).
Severe or permanent disability.
Congenital anomaly.
Or, is significant for any other reason.

Serious adverse events occurring after a patient is discontinued from the study will NOT be reported unless the investigator feels that the event may have been caused by the study drug or a protocol procedure. Study-specific clinical outcomes of death because of disease progression are exempt from serious adverse event reporting, unless the investigator deems them related to use of the study drug. Hospitalization for study drug administration is not a serious adverse event.

In general, serious adverse events assessed as clearly being due to disease progression and not due to study drug(s) should be excluded from adverse event reporting. However, in cases where the specificity or severity of an event is not consistent with the risk information, the event should be reported.

Phase I Results

Treatment

Treatment was as follows:

1 dose=a combination of docetaxel, bevacizumab, and gemcitabine

Each dose is administered intravenously every 2 weeks.

1 course=2 administered doses=4 weeks

Drug dosing:

Docetaxel: 50 mg/m²

Bevacizumab: 5 mg/kg

Gemcitabine: Increasing doses, starting at 1,000 mg/m²

Each patient cohort is studied at an increment dose of 250 mg/² of gemcitabine

The following patients were enrolled on the various cohort:

| Patient- | Gemcitabine dose | | Number of courses |
|---|---|---|---|
| Cohort | Mg/m² | Number of patients | received/patient |
| 1 | 1000 | 3 | 2, 7, 6 |
| 2 | 1250 | 3 | 4, 4, 4 |
| 3 | 1500 | 2 | 1, 1 |

Adverse Events

The side effects are shown in the table. There were no ≧grade 3 adverse events related to the treatment. Therefore, the treatment is extremely well tolerated. The most frequent adverse events are alopecia, diarrhea, fatigue, rigors, nausea, headache, and dyspnea.

Adverse Events (N = 6 patients)

| | | | | Relationship to |
|---|---|---|---|---|
| Adverse Event | Grade 1 | Grade 2 | TOTAL (%) | drug |
| Anemia | 2 | | 2 (33) | probable |
| Leukopenia | | 1 | 1 (17) | definite |
| Alopecia | 4 | 1 | 5 (83) | definite |
| Anorexia | 1 | | 1 (17) | probable |
| Chest pain | 1 | 1 | 2 (33) | possible |
| Constipation | 1 | 1 | 2 (33) | possible |
| Diarrhoea NOS | 5 | | 5 (83) | probable |
| Dysgeusia | 1 | | 1 (17) | probable |
| Dyspnea | 2 | 1 | 3 (50) | probable |
| Epistaxis | 2 | | 2 (33) | definite |
| Fatigue | 2 | 3 | 5 (83) | probable |
| Fever | 1 | | 1 (17) | possible |
| Headache | 1 | 2 | 3 (50) | probable |
| Hemorrhoids | | 1 | 1 (17) | probable |
| Hypertension | 1 | | 1 (17) | definite |
| Nail disorder | | 1 | 1 (17) | definite |
| NOS | | | | |
| Nausea | 3 | | 3 (50) | probable |
| Proteinuria | | 1 | 1 (17) | possible |
| Rash | | 1 | 1 (17) | probable |
| Rigors | 4 | | 4 (66) | probable |
| Sensory | 1 | | 1 (17) | probable |
| neuropathy | | | | |
| Stomatitis | 2 | | 2 (33) | Definite |
| Tachycardia | 1 | | 1 (17) | Possible |

Response to Treatment

There is one RECIST evidence of response for any patients. The other patients did not have evidence of RECIST criteria response, which is a phenomenon well known in the treatment of sarcoma. This means that on standard imaging procedures (CT-scan or MRI), the tumors do not appear to shrink. However, by MRI, they appear more necrotic, a potential sign of treatment activity.

Cohort 1
Patient 1: This patient (myxoid sarcoma) did not respond and had some sign of progression. He did not respond to a salvage standard regimen either (adriamycin and ifosfamide).
Patients 2 and 3: These two patients (leiomyosarcoma and liposarcoma) were having metastatic or inoperable disease. They each have received 6 and 7 courses of chemotherapy.
   The first patient had objective signs of tumor shrinkage by physical examination: subcutaneous tumors, which were proven to be metastases by fine needle aspiration and histological examination, have disappeared. The patient girth which had increased because of tumor growth inside the abdomen, decreased and he was able to fit his pants again. His energy is back to normal.
   The second patient has a confirmed partial remission.

Cohort 2

Patients 1, 2 and 3: These 3 patients (myxoid sarcoma, malignant peripheral nerve sheet tumor, and angiosarcoma) were treated in an adjuvant fashion. They received 2 courses with bevacizumab and 2 courses without bevacizumab in preparation for surgery. The 2 first tumors became necrotic by MRI imaging and both patients who were in a wheelchair prior to starting chemotherapy are now fully ambulatory. The patient with angiosarcoma had a purplish tumor who became white during treatment. None of these tumors have grown during treatment. Surgery is planned by end of August or early September for each patient. Pathology slides will be added as further documentation of response.

Cohort 3

Patients 1 and 2: Too early. Results will be available at the earliest at the end of August.

An additional 16 patients are anticipated to be added to this trial.

### REFERENCES

1. Jemal, A, Tiwari, R C, Murray, T, et al. Cancer statistics, 2004. CA Cancer J Clin 2004; 54:8.

2. Billingsley, K G, Burt, M E, Jara, E, et al. Pulmonary metastases from soft tissue sarcoma: analysis of patterns of diseases and postmetastases survival. Ann Surg 1999; 229:602.

3. Potter, D A, Glenn, J, Kinsella, T, et al. Patterns of recurrence. J Clin Oncol 1985; 3:353.

4. Pearlstone, D B, Pisters, P W, Bold, R J, et al. Patterns of recurrence in extremity liposarcoma: Implications for staging and follow-up. Cancer 1999; 85:85.

5. Spillane, A J, Fisher, C, Thomas, J M. Myxoid liposarcoma—the frequency and the natural history of nonpulmonary soft-tissue metastases. Ann Surg Oncol 1999; 6:389.

6. Mazeron, J J, Suit, H D. Lymph nodes as sites of metastases from sarcomas of osft tissue. Cancer 1987; 60:1800.

7. Van Glabbeke, M, van Oosterom, A T, Oosterhuis, J W, et al. Prognostic factors for the outcome of chemotherapy in advanced soft tissue sarcoma: an analysis of 2185 patients treated with anthracycline-containing first-line regimensa European Organization for Research and Treatment of Cancer Soft Tissue and Bone Sarcoma Group Study. J Clin Oncol 1999; 17:150.

8. Vogt-Moykopf, I, Bulzebruck, H, Merkle, N M, Probst, G. Results of surgical treatment of pulmonary metastases. Eur J Cardiothorac Surg 1988; 2:224.

9. Choong, P F, Pritchard, D J, Rock, M G, et al. Survival after pulmonary metastasectomy in soft tissue sarcoma. Prognostic factors in 214 patients. Acta Orthop Scand 1995; 66:561.

10. Keohan, M L, Taub, R N. Chemotherapy for advanced sarcoma: Therapeutic decisions and modalities. Semin Oncol 1997; 24:572.

11. Rosen, G, Forscher, C, Lowenbraun, S, et al. Synovial sarcoma. Uniform response of metastases to high dose ifosfamide. Cancer 1994; 73:2506.

12. Spillane, A J, A'Hern, R, Judson, I R, et al. Synovial sarcoma: a clinicopathologic, staging, and prognostic assessment. J Clin Oncol 2000; 18:3794.

13. Edmonson, J H, Marks, R S, Buckner, J C, Mahoney, M R. Contrast of response to dacarbazine, mitomycin, doxorubicin, and cisplatin (DMAP) plus GM-CSF between patients with advanced malignant gastrointestinal stromal tumors and patients with other advanced leiomyosarcomas. Cancer Invest 2002; 20:605.

14. Balcerzak S P, Benedetti J, Weiss G R, et al. A phase II trial of paclitaxel in patients with advanced soft tissue sarcomas: a Southwest Oncology Group study. Cancer 1995; 76: 2248-52

15. Gallup D G, Blessing J A, Andersen W, et al. Evaluation of paclitaxel in previously treated leiomyosarcoma of the uterusa gynecologic oncology group study. Gynecol Oncol 2003; 89: 48-51

16. Fata F, O'Reilly E, Ilson D, et al. Paclitaxel in the treatment of patients with angiosarcoma of the scalp or face. Cancer 1999; 1997; 38: 7

17. van Hoesel Q G, Verweij J, Catimel G, et al. Phase II study docetaxel (Taxotere) in advanced soft tissue sarcomas of adult: EORTC Soft Tissue and Bone Sarcoma Group. Ann 2002; 20: 2181-8

18. Kostler W J, Brodowicz T, Attems Y, et al. Docetaxel as rescue medication in anthracycline- and ifosfamide-resistant locally advanced or metastatic soft tissue sarcoma: results of a phase II trial. Ann Oncol 2001;

19. Edmonson J H, Ebbert L P, Nascimento A G, et al. Phase II study of docetaxel in advanced soft tissue sarcomas. Am J Clin Oncol 1996; 19: 574-6

20. Spath-Schwalbe E, Genvresse I, Koschuth A, et al. Phase II trial of gemcitabine in patients with pretreated advanced soft sarcomas. Anticancer Drugs 2000; 11: 325-9

21. Svancarova L, Blay J Y, Judson I R, et al. Gemcitabine in advanced adult soft-tissue sarcomas: a phase II study of the EORTC Soft Tissue and Bone Sarcoma Group. Eur J Cancer 2002; 38: 556-9

22. Okuno S, Edmonson J, Mahoney M, et al. Phase II tria of gemcitabine in advanced sarcomas. Cancer 2002; 94: 3225-9

23. Verweij J, Lee S M, Ruka W, et al. Randomized phase II study of docetaxel versus doxorubicin in first- and second-line sarcomas in adults: a study of the European organization for research and treatment of cancer soft tissue and bone sarcoma tissue sarcoma: a trial of the EORTC Early Clinical Studies group. J Clin Oncol 2000; 18: 2081-6

24. Okuno S, Ryan L M, Edmonson J H, et al. Phase II trial of gemcitabine in patients with advanced sarcomas (E1797): a Cancer 2003; 97: 1969-73

25. Hartmann J T, Oechsle K, Huober J, et al. An open label, non-comparative phase II study of gemcitabine as salvage treatment for patients with pretreated advanced soft tissue sarcoma J Cancer Res Clin Oncol 2004; 130:

26. Hensley M L, Maki R, Venkatraman E, et al. Gemcitabine and docetaxel in patients with unresectable leiomyosarcoma: results of a phase II trial. J Clin Oncol 2002; 20: 2824-31

27. Amodio A, Carpano S, Manfredi C, et al. Gemcitabine in advanced stage soft tissue sarcoma: a phase II study [in Italian]. Clin Ter 1999; 150: 17-20

28. Potti A, Ganti A K, Foster H, Knox S, Hebert B J, Tendulkar K, Sholes K, Koch M, Kargas S. Immunohistochemical detection of HER-2/neu, c-kit (CD117) and vascular endothelial growth factor (VEGF) overexpression in soft tissue sarcomas. Anticancer Res. 2004 January-February;24(1):333-7.

29. Hayes A J, Mostyn-Jones A, Koban M U, A'Hem R, Burton P, Thomas J M. Serum vascular endothelial growth factor as a tumour marker in soft tissue sarcoma. Br J Surg. 2004 February; 91(2):242-7.

30. Figg W D, Kruger E A, Price D K et al: Inhibition of angiogenesis: treatment options for patients with metastatic prostate cancer. Invest New Drugs 2002; 20:183-194.

31. Gordon M S, Margolin K, Talpaz M et al: Phase I safety and pharmacokinetic study of recombinant human anti-vascular endothelial growth factor in patients with advanced cancer. J Clin Oncol 2001; 19(3):843-850.

32. Hurwitz H: Bevacizumab (Avastin, a monoclonal antibody to vascular endothelial growth factor) prolongs survival in first-line colorectal cancer (CRC): results of a phase III trial of bevacizumab in combination with bolus IFL (irinotecan, 5-fluorouracil, leucovorin). Presented at the 39th Annual American Society of Clinical Oncology Meeting, Chicago, Ill.; 2003.

33. Cobleigh M A, Miller K D, Langmuir V K et al: Phase II dose escalation trial of Avastin(™) (bevacizumab) in women with previously treated metastatic breast cancer (abstract 520). Breast Cancer Res Treat 2001; 69(3):301.

34. Chen H X, Gore-Langton R E & Cheson B D (ed): Clinical trials: referral resource: current clinical trials of the anti-VEGF monoclonal antibody bevacizumab. Oncology 2001; 15(8):1019-1026.

35. Simon R, Freidlin B, Rubinstein L, Arbuck S G, Collins J, Christian M C. Accelerated titration designs for phase I clinical trials in oncology. J Natl Cancer Inst. 1997 Aug. 6; 89(15):1138-47.

36. Kaplan El, Meier P. Non parametric estimation from incomplete observations. J Am Stat Assoc 53:457-481, 1958

1. A method of treating cancer comprising administering to a patient in need of therapy an effective amount of low dose, frequently administered combination of gemcitabine, docetaxel and an angiogenesis inhibitor.

2. The method according to claim 1 wherein said angiogenesis inhibitor is selected from the group consisting of ZD6474, ZD 6126, AZD2171, SU6668 and SU5416, bevacizumab, mv833, anti-FLT-1 ribozyme, SU5416, PTK 787, ZD4190, ZD6474, CEP-7055, SU11248 and mixtures thereof.

3. The method according to claim 1 wherein said cancer is sarcoma and said inhibitor is bevacizumab.

4. The method according to claim 2 wherein said sarcoma is a soft tissue sarcoma.

5. The method according to claim 4 wherein said soft tissue sarcoma is selected from the group consisting of fibrosarcoma, malignant fibrous hystiocytoma, liposarcoma, rhabdomyosarcoma, leiomyosarcoma, hemangiosarcoma, Kaposi's sarcoma, lymphangiosarcoma, synovial sarcoma, neurofibrosarcoma, extraskeletal chrondrosarcoma, extraskeletal osteosarcoma, embryonal sarcoma, alveolar sarcoma, dermatofibrosarcoma, infantile heamangiopericytoma, malignant peripheral nerve sheath tumors, alveolar soft part sarcoma, extraskeletal myxoid chondrosarcoma, and extraskeletal mesenchymal sarcoma.

6. The method according to claim 1 wherein the treatment results in one or more of clinical benefit remission, an increased quality of life or prolongation of survival of the patient.

7. The method according to claim 1 wherein said treatment results in the shrinkage of a tumor or prolonged stability of the cancer.

8. The method according to claim 1 wherein said treatment reduces metastases of said cancer.

9. A pharmaceutical composition comprising an effective amount of a combination of gemcitabine, docetaxel and an angiogenesis inhibitor.

10. The composition according to claim 9 wherein said angiogenesis inhibitor is selected from the group consisting of ZD6474, ZD 6126, AZD2171 (Astra Zeneca), SU6668 and SU5416 (Sugen), bevacizumab (Avastatin), mv833, anti-FLT-1 ribozyme (Angiozyme), and the tyrosine kinase inhibitors SU5416 (Semaxanib), PTK 787 (ZK 222584), ZD4190, ZD6474, CEP-7055, SU11248 and mixtures thereof.

11. The composition according to claim 9 wherein said inhibitor is bevacizumab.

12. The composition according to claim 9 adapted for parenteral administration.

13. The composition according to claim 12 adapted for intravenous administration.

14. A method for treating a cancer patient with soft tissue sarcoma, wherein said soft tissue sarcoma is unresponsive to traditional therapy, said method comprising administering to said patient a combination of gemcitabine, docetaxel and an angiogenesis inhibitor in amounts effective to provide a clinical benefit remission, an increased quality of life or prolongation of survival of the patient.

15. The method according to claim 13 wherein said treatment results in the shrinkage of a tumor or prolonged stability of the cancer.

16. The method according to claim 13 wherein said method results in a complete remission of said soft tissue sarcoma.

17. The method according to claim 16 wherein said angiogenesis inhibitor is selected from the group consisting of ZD6474, ZD 6126, AZD2171 (Astra Zeneca), SU6668 and SU5416 (Sugen), bevacizumab (Avastatin), mv833, anti-FLT-1 ribozyme (Angiozyme), and the tyrosine kinase inhibitors SU5416 (Semaxanib), PTK 787 (ZK 222584), ZD4190, ZD6474, CEP-7055, SU11248 and mixtures thereof.

18. The method according to claim 17 wherein said angiogenesis inhibitor is bevacizumab.

19. The method according to claim 14 wherein said soft tissue sarcoma is selected from the group consisting of fibrosarcoma, malignant fibrous hystiocytoma, liposarcoma, rhabdomyosarcoma, leiomyosarcoma, hemangiosarcoma, Kaposi's sarcoma, lymphangiosarcoma, synovial sarcoma, neurofibrosarcoma, extraskeletal chrondrosarcoma, extraskeletal osteosarcoma, embryonal sarcoma, alveolar sarcoma, dermatofibrosarcoma, infantile heamangiopericytoma, malignant peripheral nerve sheath tumors, alveolar soft part sarcoma, extraskeletal myxoid chondrosarcoma, and extraskeletal mesenchymal sarcoma.

20. The method according to claim 19 wherein said prolongation of survival is at least 2 years.
